(19) United States
(12) Patent Application Publication
(10) Pub. No.: US 2023/0338602 A1
(43) Pub. Date: Oct. 26, 2023

(54) AROMA DIFFUSING DEVICE

(71) Applicant: HUAN-PING LEE, Toufen City (TW)

(72) Inventor: HUAN-PING LEE, Toufen City (TW)

(21) Appl. No.: 17/726,546

(22) Filed: Apr. 22, 2022

(51) Int. Cl. A61L 9/03

(52) U.S. Cl. CPC A61L 9/03; A61L 2209/111; A61L 2209/12

(57) ABSTRACT

The aroma diffusing device includes a base having an accommodation space inside, a heating member in the accommodation space having a heater unit and a power control unit, and a tray. The heater unit is electrically connected to the power control unit and exposed out of the base. The tray is mounted on the base contacting with the heater unit. The tray holds an aromatic essential oil which is heated by the heater unit under the control of the power control unit.

### BACKGROUND OF THE INVENTION

### (a) Technical Field of the Invention

The present invention is generally related to aroma diffusing device, and more particular to an aroma diffusing device that may also function as a lamp.

### (b) Description of the Prior Art

Aromatherapy is widely popular in these days for relieving stress. Aromatherapy is a form of alternative therapy where a user absorbs aromatic materials, such as essential oils, through message or bath to improve psychological of physical well-being. The essential oils extracted from different plants are claimed to have different effects. Currently, there are aroma diffusers heats and evaporates the essential oil for the user to breathe in and, as such, to relax.

The conventional aroma diffusers have the following shortcomings.

Firstly, the aroma diffusers commonly employ a halogen lamp for heating. The lamp would produce surplus heat, is more hazardous, and consumes more power.

Secondly, if a lamp is employed for both lighting and heating essential oil, the light may interfere a user's sleep. If the lamp is not used for heating essential oil, a separate mechanism is required for heating the essential oil.

### SUMMARY OF THE INVENTION

To obviate the above shortcomings, the present invention discloses an aroma diffusing device including a base having an accommodation space inside, a heating member in the accommodation space having a heater unit and a power control unit, and a tray. The heater unit is electrically connected to the power control unit and exposed out of the base. The tray is mounted on the base contacting with the heater unit. The tray holds an aromatic essential oil which is heated by the heater unit under the control of the power control unit.

The foregoing objectives and summary provide only a brief introduction to the present invention. To fully appreciate these and other objects of the present invention as well as the invention itself, all of which will become apparent to those skilled in the art, the following detailed description of the invention and the claims should be read in conjunction with the accompanying drawings. Throughout the specification and drawings identical reference numerals refer to identical or similar parts.

Many other advantages and features of the present invention will become manifest to those versed in the art upon making reference to the detailed description and the accompanying sheets of drawings in which a preferred structural embodiment incorporating the principles of the present invention is shown by way of illustrative example.

### BRIEF DESCRIPTION OF THE DRAWINGS

FIG. 1 is a perspective diagram showing an aroma diffusing device according to a first embodiment of the present invention.

FIG. 2 is a functional block diagram showing the aroma diffusing device of FIG. 1.

FIG. 3 is a perspective diagram showing an aroma diffusing device according to a second embodiment of the present invention.

FIG. 4 is a perspective diagram showing an aroma diffusing device according to a third embodiment of the present invention.

FIG. 5 is a perspective diagram showing an aroma diffusing device according to a fourth embodiment of the present invention.

FIG. 6 is another perspective diagram showing the aroma diffusing device of FIG. 5.

### DETAILED DESCRIPTION OF THE PREFERRED EMBODIMENTS

The following descriptions are exemplary embodiments only, and are not intended to limit the scope, applicability or configuration of the invention in any way. Rather, the following description provides a convenient illustration for implementing exemplary embodiments of the invention. Various changes to the described embodiments may be made in the function and arrangement of the elements described without departing from the scope of the invention as set forth in the appended claims.

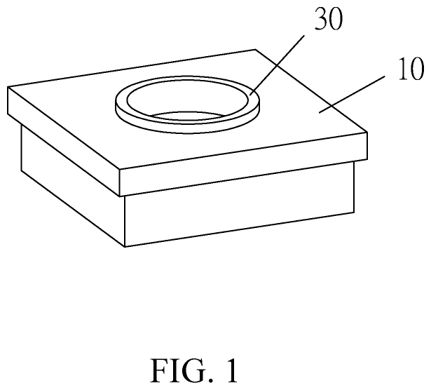

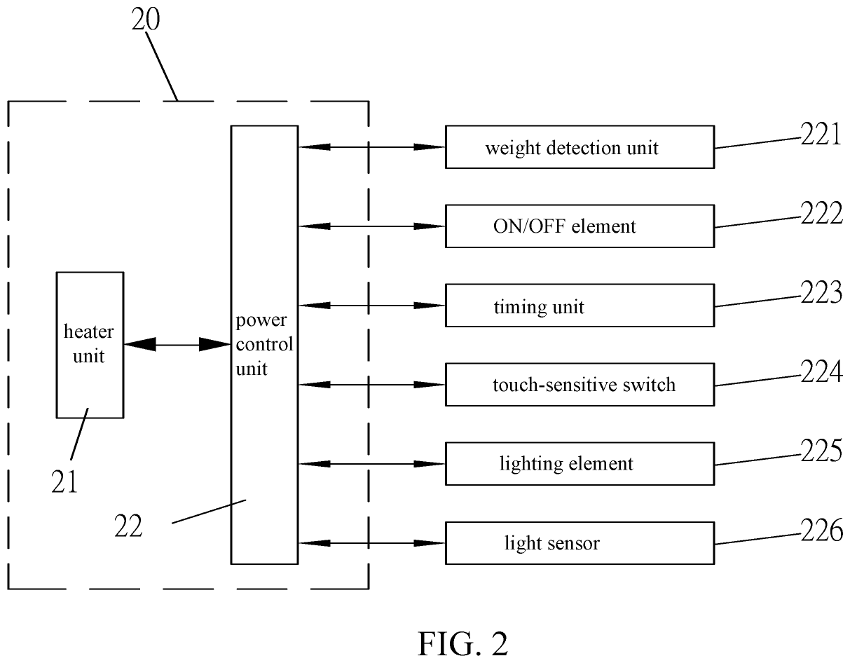

As shown in FIGS. 1 and 2, an aroma diffusing device according to a first embodiment of the present invention includes:
a base 10 having an accommodation space inside (not shown) and a top opening on a top side exposing the accommodation space;
a heating member 20 in the accommodation space, including a heater unit 21 and a power control unit 22, where the heater unit 21 is electrically connected to the power control unit 22, and the heater unit 21 is exposed from the top opening of the base 10; and
a tray 30 detachably mounted on the top opening of the base 10 contacting with the heater unit 21 through the top opening of the base 10;
wherein the tray 30 holds a fragrant or aromatic essential oil which is heated by the heater unit 21 under the control of the power control unit 22.

To enhance the efficiency and safety of the aroma diffusing device, the aroma diffusing device further includes
a weight detection unit 221 electrically connected to the power control unit 22, where the power control unit 22 activates or deactivates the heater unit 21 when the weight detection unit 221 detects that a weight of the essential oil in the tray 30 is greater or less than a preset weight value;
a ON/OFF element 222 and a timing unit 223 electrically connected to the power control unit 22, where the ON/OFF element 222 turns on the heater unit 21 and automatically turns off the heater unit 21 after being triggered by the timing unit 223 after a preset duration after the heater unit 21 is turned on; and
a touch-sensitive switch 224 configured on the base 10 and electrically connected to the power control unit 22, where, once touched, the touch-sensitive switch 224 turns on or off the heater unit 21.

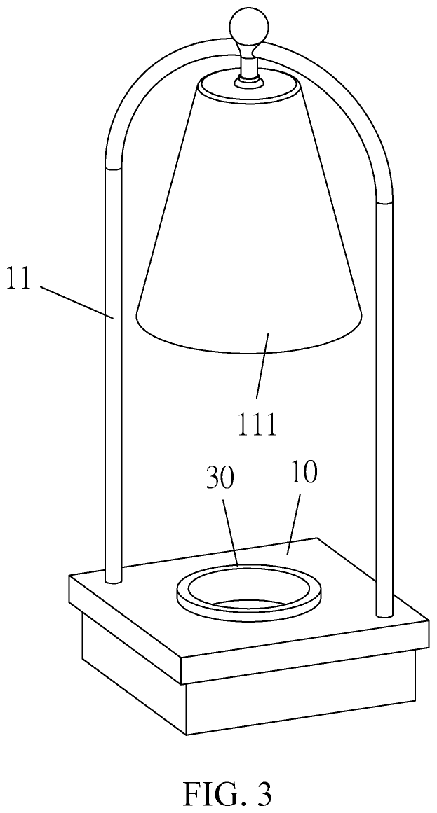

As shown in FIG. 3, a second embodiment of the present invention further includes a stand 11 mounted on the top side of the base 10. A lighting element 225, such as a light emitting diode (LED), is configured on the stand 11 and electrically connected to the power control unit 22 so that the aroma diffusing device can also function as a lamp. The stand 11 may be further configured with a shade 111 and the lighting element 225 is positioned inside the shade 111. The shade 111 may be configured with a second touch-sensitive switch 224 electrically connected to the power control unit 22 where, once touched, the lighting element 225 is turned on or off.

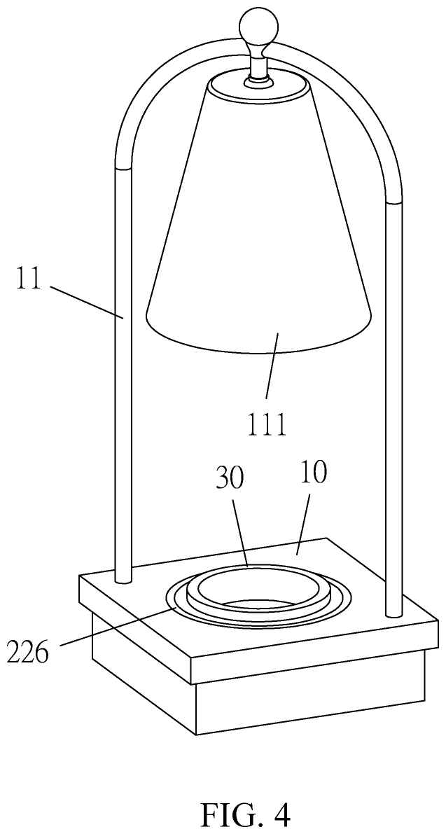

As shown in FIG. 4, a third embodiment of the present invention further includes a light sensor 226 configured on the base 10 electrically connected to the power control unit 22, where the light sensor 226 turns on the heater unit 21 when detecting light from the lighting element 225.

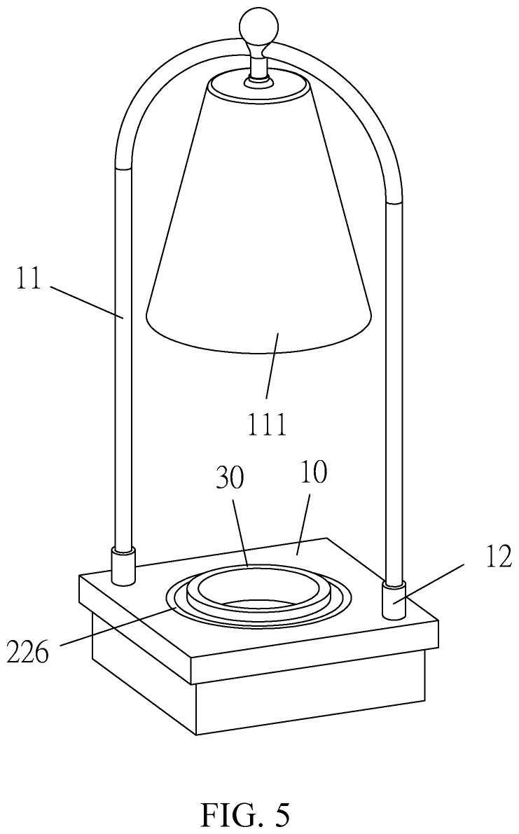

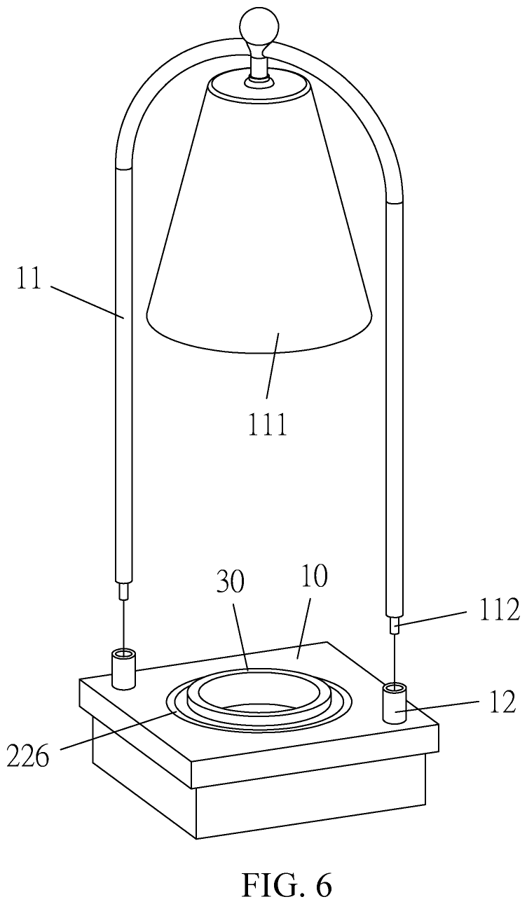

As shown in FIGS. 5 and 6, a fourth embodiment of the present invention further includes a number of first connectors 12 on the top side of the base 10 and a number of second connectors 112 on the stand 11. The second connectors 112 may be detachably joined to the first connectors 12. As such, stands 11 and shades 111 of different styles may be fitted on the aroma diffusing device. As the lighting element 225 is configured on the stand 11, lighting elements 225 of different color or brightness may be switched as well.

While certain novel features of this invention have been shown and described and are pointed out in the annexed claim, it is not intended to be limited to the details above, since it will be understood that various omissions, modifications, substitutions and changes in the forms and details of the device illustrated and in its operation can be made by those skilled in the art without departing in any way from the claims of the present invention.

I claim:

1. An aroma diffusing device, comprising:
a base having an accommodation space inside;
a heating member in the accommodation space comprising a heater unit and a power control unit, where the heater unit is electrically connected to the power control unit, and the heater unit is exposed out of the base;
a tray positioned on the base contacting with the heater unit; and
a stand mounted on the base and a lighting element configured on the stand and electrically connected to the power control unit;
wherein the lighting element is a light emitting diode (LED); and the tray holds an aromatic essential oil which is heated by the heater unit under the control of the power control unit.

2. An aroma diffusing device, comprising:
a base having an accommodation space inside;
a heating member in the accommodation space comprising a heater unit and a power control unit, where the heater unit is electrically connected to the power control unit, and the heater unit is exposed out of the base; and
a tray mounted on the base contacting with the heater unit;
wherein the tray holds an aromatic essential oil which is heated by the heater unit under the control of the power control unit.

3. The aroma diffusing device according to claim 2, wherein the base has a top opening on a top side exposing the accommodation space; and the tray is detachably positioned in the top opening.

4. The aroma diffusing device according to claim 2, further comprising a weight detection unit electrically connected to the power control unit, where the power control unit activates or deactivates the heater unit when the weight detection unit detects that a weight of the essential oil in the tray is greater or less than a preset weight value.

5. The aroma diffusing device according to claim 2, further comprising a ON/OFF element and a timing unit, both electrically connected to the power control unit, where the ON/OFF element turns on the heater unit and automatically turns off the heater unit after being triggered by the timing unit after a preset duration after the heater unit is turned on.

6. The aroma diffusing device according to claim 2, further comprising a touch-sensitive switch configured on the base and electrically connected to the power control unit, where, once touched, the touch-sensitive switch turns on or off the heater unit.

7. The aroma diffusing device according to claim 2, further comprising a stand mounted on the base and a lighting element configured on the stand and electrically connected to the power control unit.

8. The aroma diffusing device according to claim 7, further comprising a light sensor configured on the base electrically connected to the power control unit, where the light sensor turns on the heater unit when detecting light from the lighting element.

9. The aroma diffusing device according to claim 7, wherein the stand is configured with a shade; and the lighting element is positioned inside the shade.

10. The aroma diffusing device according to claim 7, wherein a plurality of first connectors are configured on the base and a plurality of second connectors are configured on the stand; and the second connectors are detachably joined to the first connectors.

11. The aroma diffusing device according to claim 9, wherein the shade is configured with a second touch-sensitive switch electrically connected to the power control unit where, once touched, the lighting element is turned on or off.

12. The aroma diffusing device according to claim 9, wherein the lighting element is a light emitting diode (LED).
